(19) United States
(12) Patent Application Publication
(10) Pub. No.: US 2022/0065842 A1
(43) Pub. Date: Mar. 3, 2022

(54) Plural individual specimen collecting, transporting & analyzing device and method

(71) Applicant: Phuong Nguyen, San Diego, CA (US)

(72) Inventor: Phuong Nguyen, San Diego, CA (US)

(21) Appl. No.: 17/006,827

(22) Filed: Aug. 29, 2020

(51) Int. Cl. G01N 33/487; G01N 1/02; B01L 3/00

(52) U.S. Cl. CPC G01N 33/487; G01N 1/02; G01N 2001/027; B01L 3/5029; B01L 3/5082

(57) ABSTRACT

A device for collecting, shipping and analyzing a plurality of pathological specimens includes a plural number of collection vials for loading into testing vessel. Each vial can contain a reagent solution and a collector stick projecting from the inside of a stopper. The bottom of the vial can have a drain sealed by a frangible barrier. The vials can be inserted into chambers in the vessel that have a pintles oriented to break the barriers, allowing solution to flow onto one or more signaling elements such as a chromatographic strips. An insert drives the vials onto the pintles by simply screwing the vessel lid to its farthest position. The insert includes pins oriented to puncture sealed holes in the stoppers of each vial, facilitating the flow of solution from the vials. The analytical process can be performed in the absence of any contacting or manipulating of the vials and specimen collectors.

### FIELD OF THE INVENTION

The invention relates to immunoassay devices for testing liquid and liquid immersible specimens, and more particularly to devices for collection, storage, and preliminary screening of materials such as pathological, forensic, and/or environmental specimens.

### BACKGROUND

Liquid specimen testing containers are commonly used to collect and test liquid specimens for the presence or absence of specific “indicators” which show the presence of certain chemicals, hormones, antibodies or antigens associated with various physiological conditions and are commonly used for drug abuse screening. Such containers can also be adapted to test semi-solid material specimens such as bodily excretions, gels, and powders by mixing the specimen with one or more liquid reagents within the container.

Biological specimen testing, especially the testing of fecal samples is commonly used for detecting, diagnosing and monitoring a variety of diseases. For example, devices such as shown in Nguyen, U.S. Pat. No. 7,981,054 provide for testing fecal material specimens among other possible specimens. Nasal mucous and saliva swabs can be used to test for abused drugs and the present of viral infection such as COVID-19. In most cases, a first instrument such as a stick, spoon, or swab is used to collect a sample which is then packaged into a special enclosure for handling or shipping. In the laboratory, the specimen is usually diluted into a reagent solution where it is exposed to a mobilized binding member, conjugated to visually detectable label such as colloidal gold, that can bind to an analyte in the specimen which is specific to the condition being tested. Bound analytes are then analyzed by accumulation within a chromatographic immunoassay strip or the like having a region of immobilized binding members which signal the test result by the accumulation of labeled specimen thereon.

Devices such as those shown in Nguyen, U.S. Pat. No. 7,981,054 provide important advantages including the option of immediate, rapid testing, avoiding the handling of diverse containers and testing instruments, simplifying the testing procedure, and reducing the risk of contamination to the specimens, and more importantly contamination to the technician conducting the test. However, such devices are designed to contain and test a single specimen. Large scale rapid testing of a large number of specimens has gained urgency in light of the recent COVID-19 pandemic. Sometimes, in situations such as COVID-19 testing where a very high percentage of negative result testing is anticipated, a plural number of specimens are combined, and a single test carried out on the combined specimens.

Increasingly, preliminary screening tests are being performed and evaluated by relatively unskilled technicians or even the general public. Therefore, preliminary rapid screening devices need to be relatively simple to operate to ensure adequate exposure of the testing strips and to provide more consistent results.

Another potential problem involves the timing of the initiation of the test. Often, the results of the preliminary screening test may be valid for a narrow span of time. Thus, it can be useful to prevent the donor or others from initiating the preliminary screening test prematurely.

Therefore there is a need for a specimen test device which addresses some or all of the above identified inadequacies.

### SUMMARY

The principal and secondary objects of the invention are to help provide an improved specimen collection, optional storage and preliminary screening device. These and other objects are achieved by a vessel having a plural number collection vials that can be tested together.

In some embodiments the invention allows for triggering and performance of the analyzing process on a plural number of specimens by simply manipulating the lid of a vessel containing specimen-containing vials without having to open the vessel or contact its contents.

In some embodiments there is provided a self-contained device for collecting, transporting and analyzing a plurality of biological specimens, said device comprises: an outer vessel having a top opening, a lateral wall and a closed bottom; a hand-manipulable lid releasably sealing said top opening; at least one signaling element contained within said vessel; a plurality of pintles extending upwardly from said closed bottom; a plurality of vials housed inside said outer vessel; wherein a first one of said vials contains a first liquid specimen; wherein a second one of said vials contains a second liquid specimen; an insert contained within said vessel; wherein said insert pushes all of said vials onto said pintles upon manipulation of said lid, thereby substantially simultaneously puncturing all of said vials and releasing at least one of said liquid specimens onto said at least one signaling element.

In some embodiments the device further comprises: said first one of said vials comprising: an open upper end; a stopper releasably sealing said open upper end; a hole in said stopper and a frangible barrier closing said hole; a bottom end; a drain in said bottom end and a removable barrier closing said drain; and, a first one of said pintles being oriented to break said removable barrier upon manipulation of said lid.

In some embodiments the device further comprises: a plurality of pins extending downwardly from said insert; wherein a first one of said pins is oriented to break said frangible barrier upon manipulation of said lid.

In some embodiments said lid comprises a protrusion having a substantially planar lower surface bearing against a substantially planar upper surface of said insert, whereby said lid can rotate with respect to said insert while driving said insert axially.

In some embodiments the device further comprises a framework within said vessel defining a plurality of compartments, wherein each compartment can carry a single one of said vials.

In some embodiments said first one of said vials is coaxially aligned with a first one of said pins and a first one of said pintles.

In some embodiments said first one of said vials further comprises: a specimen collector projecting from an under portion of said stopper down to a lower region of said first one of said vials.

In some embodiments said lid comprises a disableable preventer structure preventing said lid from being fully screwed upon said vessel, and thereby preventing initiation of any puncturing of said vials until said preventer structure is disabled.

In some embodiments said first one of said pintles comprises a standoff positioned, shaped and dimensioned to keep a surface section of said closed bottom apart from a surface section of said first one of said vials.

In some embodiments there is provided a specimen collection and examination device which comprises: a first vial comprising a first specimen; a second vial comprising a second specimen; a vessel nesting said first and second vials, said vessel having an open top and a closed bottom; at least one signaling element held in said vessel; a lid sealing said open top; and, a puncturing structure within said vessel; wherein said puncturing structure punctures said vials upon manipulation of said lid.

In some embodiments the device further comprises: said first vial comprising: a first open upper end and a first closed bottom end; a first stopper shaped and dimensioned to seal said first upper end; said first bottom end having a first aperture and a first removable barrier sealing said first aperture; said second vial comprising: a second open upper end and a second closed bottom end; a second stopper shaped and dimensioned to seal said second upper end; said second bottom end having a second aperture and a second removable barrier sealing said second aperture; and, wherein said puncturing structure comprises at least two pintles extending upwardly from said closed bottom in alignment with said first and second apertures.

In some embodiments the device further comprises: an insert contained within said vessel; said first stopper comprising a first hole and a first frangible barrier sealing said first hole; said second stopper comprising a second hole and a second frangible barrier sealing said second hole; a first pin and a second pin projecting from an under section of said insert in alignment with said first and second holes.

In some embodiments said frangible barriers and said removable barriers are simultaneously broken by manipulation of said lid.

In some embodiments the device further comprises a standoff positioned, shaped and dimensioned to keep an outer surface section of said closed bottom apart from an inner surface section of said first vial.

In some embodiments there is provided a method for simultaneously analyzing a plurality of biological specimen containers, said method comprises: selecting a vessel having a top opening, a lateral wall and a closed bottom enclosing an inner chamber; loading a plurality of specimen containers into said inner chamber; rotating a hand-manipulable threaded lid onto said top opening; wherein said rotating comprises: puncturing simultaneously said specimen containers; and, allowing a flow of liquid solutions carrying specimens onto one or more test signaling elements.

In some embodiments said puncturing comprises: moving said specimen containers axially onto a plurality of pintles within said inner chamber; and driving an insert having a plurality of pins axially against said specimen containers.

In some embodiments said driving comprises: sliding a lower planar surface of said lid against an upper planar surface of said insert.

The original text of the original claims is incorporated herein by reference as describing features in some embodiments.

### BRIEF DESCRIPTION OF THE DRAWINGS

FIG. 1 is a diagrammatic, exploded, perspective view of a testing device according to an exemplary embodiment of the invention.

FIG. 2 is a diagrammatic cross-sectional side view of one of the collector vials of the device of FIG. 1.

FIG. 3 is a diagrammatic cross-sectional side view of the assembled device of FIG. 1 after testing has been initiated.

FIG. 4 is a diagrammatic top plan view of the vessel of FIG. 1 without vials.

FIG. 5 is a diagrammatic, perspective view of the underside of the insert of FIG. 1.

FIG. 6 is a diagrammatic top plan view of an alternate embodiment of the vessel for combined specimen screening.

FIG. 7 is a diagrammatic cross-sectional side view of an alternate embodiment of a vial for use with a nasal mucous swab.

FIG. 8 is a flow diagram of method steps according to an exemplary embodiment of the invention.

### DESCRIPTION OF THE EXEMPLARY EMBODIMENTS

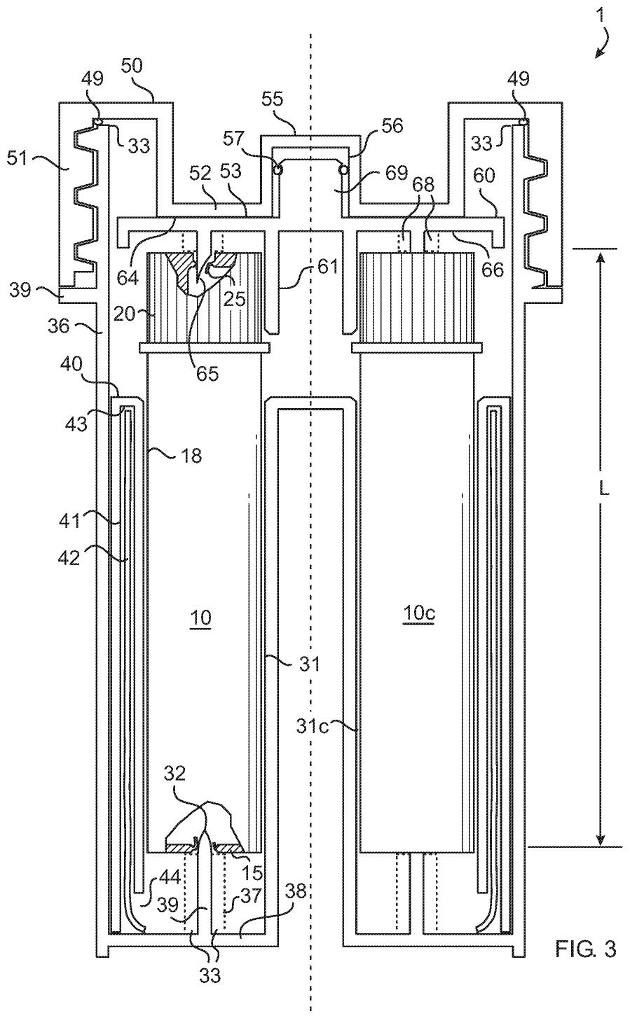

In this specification, the references to top, bottom, upward, downward, upper, lower, vertical, horizontal, sideways, lateral, back, front, etc. can be used to provide a clear frame of reference for the various structures with respect to other structures while the testing device is in its upright orientation as shown in FIG. 3, and not treated as absolutes when the frame of reference is changed, such as when the device is laying on its side.

The term “substantially” can be used in this specification because manufacturing imprecision and inaccuracies can lead to non-symmetricity and other inexactitudes in the physical characteristics, shape, dimensioning and orientation of various structures. Further, use of “substantially” in connection with certain geometrical shapes, such as “cylindrical”, “conical”, and “circular”, physical characteristics, such as “transparent”, and orientations, such as “axial”, “parallel” and “perpendicular”, can be given as a guide to generally describe the function of various structures, and to allow for slight departures from exact mathematical geometrical shapes, physical characteristics, and orientations, while providing adequately similar function. Those skilled in the art will readily appreciate the degree to which a departure can be made from the mathematically exact geometrical references or descriptions.

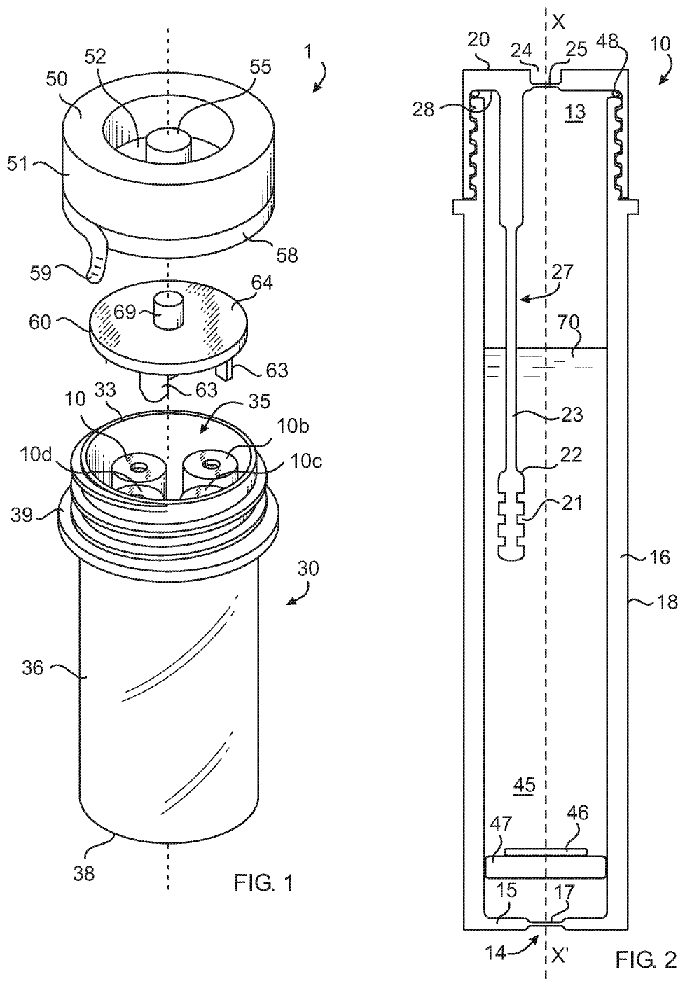

Referring now to the drawing, there is shown in FIGS. 1-5, a self-contained device 1 for collecting, transporting and analyzing a plurality of separate biological specimens. In this embodiment, the device is specifically designed for use in connection with fecal specimens but could be readily adapted to collect saliva, nasal mucous, other bodily fluids or various other materials such as food for contamination testing, or dust for the presence of illegal substances, for example. It shall be noted that a cross sectional view may be taken on multiple planes in order to enhance the diagrammatic nature of the drawing.

The device can include a plurality of inner tubular enclosures, containers or vials 10,10b,10c,10d. Each vial, 10 for example, can have a substantially tubular body 16 having a bottom end 15 and an open upper end 13 sealed by a stopper 20. An aperture forming a drain 14 in the closed bottom of the vial can be sealed by a removable barrier 17. That barrier may be constituted by a frangible web of glass, foil or plastic of the same material as the body. The vial can contain a liquid solution 70. A liquid permeable filter 47 can be installed in the bottom region 45 of the each vial. A dried deposit 46 of a mobilizable binding member specific to an analyte in question may be placed above the filter. Alternately, the mobilizable binding member can be predissolved in the solution.

A specimen collector 27 can project downwardly from and under portion 28 of the stopper 20. The collector can have a shaft 23 which terminates at its tip into a head 22 having a plurality of indentations 21. A fecal sample can be collected by plunging the head into fecal matter of which small volumes will be retained by the indentations 21. Once the stopper is installed upon the body 16, and the open upper end sealed, the vial 10 can then be inserted and completely housed in one of a plurality of uniformly, angularly spaced apart compartments 31,31b,31c,31d within the internal chamber 35 of an outer tubular enclosure, or vessel 30. Additional vials 10b,10c,10d can be constructed similarly to the vial 10, and can be similarly inserted and completely housed in the other compartments.

The vessel 30 can have a substantially transparent, substantially cylindrical lateral wall 36 extending upwardly from a closed bottom 38 and terminating at a top opening 33 to form the internal chamber 35. The top opening 33 of the vessel can be sealed by a hand-manipulable lid 50 having an internally threaded peripheral flange 51 that screws over the externally threaded lateral wall adjacent to the top opening.

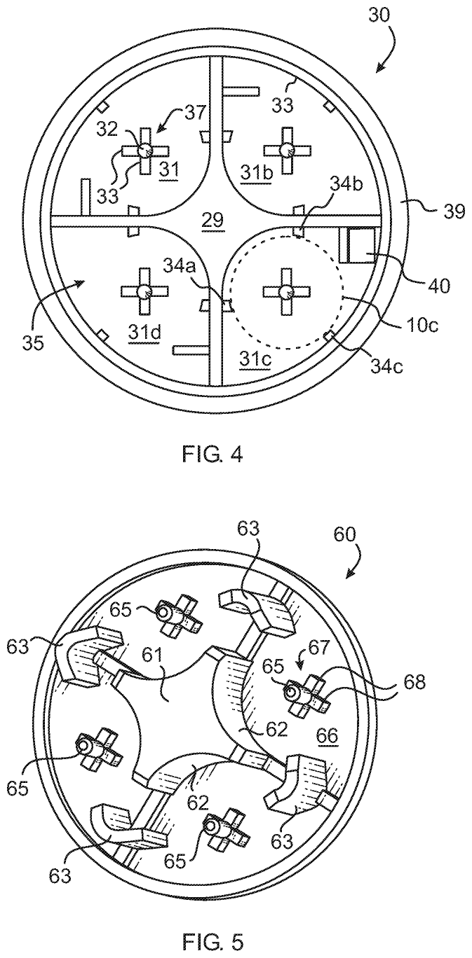

As shown primarily in FIG. 4, the compartments 31,31b,31c,31d can be formed by an internal framework 29 occupying part of the internal chamber 35 of the vessel 30. The framework can include a number of uniformly angularly spaced apart guides 34a-34c to nest each vial, 10c for example, and secure its position within its respective compartment 31c.

Each compartment, 31 for example, can include a substantially transparent cartridge 40 shaped and dimensioned to extend up the inner surface of the lateral wall 36 of the vessel 30 and form at least one pocket 41 for holding at least one signaling element in the form of a chromatographic immunoassay strip 42 in an upright orientation against the substantially transparent inner wall for convenient viewing of a test result. Each pocket terminates at a closed upper end 43 to help restrict axial movement of the strip, and a lower end gap 44 for allowing solution from the vessel to contact the bottom portion of the strip. The cartridge can be conveniently molded from a unitary piece of plastic material so that a plural number of adjacent pockets can be formed accommodating a plurality of strips. The cartridge can be positioned to keep the outer wall surfaces 18 of the vial 10 apart from the inner wall surface 36 of the vessel.

Each compartment, 31 for example, can further include a structure for puncturing a vial loaded into the compartment. The puncturing structure can include a standoff 37 extending upwardly from the bottom 38 of the vessel 30 and below the bottom end 15 of the vial 10. The standoff can include a post 39 from which radiates four arms 33 in a quadrangular pattern. A pintle 32 projects upwardly from the post in alignment with the drain 14 of the vial so that it can puncture and thus break the barrier 17 when the vial is forcibly pushed all the way down into the compartment. When this occurs, the solution 70 in the vial partially flows into the bottom of the compartment, then on through the gaps 44 in the cartridge 40, and contact the bottom portion of the test strips 42. The standoff separates a surface section on the bottom end of the vial from a surface section at the bottom of the vessel in order to provide an unobstructed path for liquid to flow from the vial to the strip or strips. Alternately the standoff can be formed from a piece of material inserted into the bottom of each chamber. Care must be taken however to ensure fluid communication between the pintle and the strip or strips. The coaxial alignment of the drain with the pintle is assured by shaping and dimensioning of the framework 29 forming the compartments.

In order to equilibrate the pressure within the vials, 10 for example, and the vessel 30, and facilitate the flow of the solution 70 from vials into the vessel, the stopper 20 of each vial can have a hole 24 sealed by a frangible barrier 25 of the same type as the bottom barrier 31. The specimen collector 17 can be conveniently located at a position offset from the hole 24 so that the hole is not blocked.

The device can include an insert 60 shaped and dimensioned to coaxially engage the upper opening 33 of the vessel 30. The insert can include a plurality of pins 65 projecting downwardly from an under surface section 66 of the insert toward the holes 24 and frangible barriers 25 in each of the vials loaded into the vessel. Each pin can include a standoff 67 having four radiating arms 68 in a quadrangular pattern. The insert can include a central pedestal 61 having concave, arcuate sidewalls 62, corresponding to each compartment, to bear against the commensurately convex sides of the stoppers 20 of the loaded vials 10 and thereby help align the insert to the stoppers. The insert can further include a number of downwardly projecting spacers 63 dimensioned and oriented to maintain the spacing between the stoppers while the insert is being driven onto the stoppers during the breaking of the barriers.

The shape and dimensioning of the vials, compartments, and puncturing structure can be uniform so that puncturing of the vials occurs substantially simultaneously. In other words, the drains and the and the holes of all the vials can be opened at substantially the same time in order to allow flow of solution out of the vials so that the tests on all the specimens in the vessel can occur substantially simultaneously. The alignment of the pins 65 with the holes 24, and the pintles 32 with the drains 14 results from the fact these various elements are precisely located with respect to the vertical axis X-X′ of the device.

The downward pressure of the lid 50 can be applied to the insert 60 by a substantially disk-shaped protrusion 52 having a substantially planar lower surface 53 formed in a medial portion of the lid. The lower surface slidingly bears against a substantially planar upper surface 64 of the insert. The lid and insert remain aligned during the twisting of the lid by a rotational bearing structure 55 including a cylindrical well 56 rotatively engaged by a corresponding cylindrical axle 69 extending upwardly from the center of the insert. The bearing structure allows relative angular motion between the insert and the lid during rotation of said lid with respect to the vessel. In other words, the insert can move axially without rotating so that alignment of the pins is maintained. An optional resilient O-ring 57 can secure the insert to the underside of the lid while allowing relative rotational movement. If the O-ring is used, the spacers 63 are relied upon to angularly orient the insert to the vials as the lid is initially screwed onto the vessel. Alternately, if the O-ring is not used, the insert can be inserted into the vessel in angular alignment prior to screwing on the lid.

Additional O-rings 48 and 49 can be mounted to the stopper and the lid in order to provide a hermetic closure. Alternately, the tolerances and resiliency of the materials of the components can be selected to provide an adequate seal.

The lid can be prevented from being screwed to its farthest position by a disableable preventer structure in the form of a base ring 58 which is formed integrally with the lid which bears against an outer peripheral shelf 39 of the vessel 30. A pull-tab 59 can allow for convenient peeling off of the base ring from the lower edge of the flange 51 thus disabling it from preventing further screwing of the lid and puncturing the vials.

When the lid 50 is only partially screwed upon the vessel 30, the pintles 32 remain away from the removable barriers 17 at the bottom of each of the vials 10, and the pins 65 remain separated from the frangible barriers 25 in the stoppers 20 of each of the vials. When the lid 50 is fully screwed upon the outer vessel opening, the pintles 32 and the pins 65 break their respective barriers with which they are aligned. The length L of the vial is selected so that proper spacing exists between the pin and pintles in both positions.

Once a specimen has been collected with the stopper 20 and collector 17 combination, it is dipped into the solution 70 by screwing the stopper over upper open end 13 of the vial 10. The vial can then be shipped to a laboratory for analysis where it can be inserted into the vessel kept at the lab. Once the vial is inserted into the vessel 30, the lid 50 is placed over the top opening of the outer vessel and screwed as far as the preventer ring 58 will allow. The device can then be briefly stored until the other compartments are loaded with vials, or analysis is initiated. In this way, a person conducting the test can have greater protection from contamination. For example one person wearing gloves can preload the vials into the vessel, while a second, less protected worker can initiate the test. The analysis of the specimen can be performed without contacting or manipulating the inner vessel or the collector. The analysis is initiated by peeling off the preventer ring 58 and screwing the lid 50 as far as it will go. Upon puncture of the barriers 17 and 25, the solution 70 containing particles of the specimen processed by exposure to mobilized binding members is filtered and partially flows into the bottom of the vessel 30 through the gaps formed by the standoffs 37, and to contact the immunoassay strips 42.

It has been found that for a substantially cylindrical vessel 30 housing substantially cylindrical vials 10, four compartments provides adequately efficient utilization of the available space within the vessel while limiting the amount of torque necessary to break all eight barriers substantially simultaneously, as can be easily achieved manually using the relatively large diameter lid. Further, such torque is transmitted to the downward force on the barriers in a controlled manner.

An advantage of the above described device is that it can be readily adapted to provide aggregate testing of plural samples by combining the outflow of the chambers into a single sump in which plural solutions are mixed and fed to a signaling device such as a chromatographic strip.

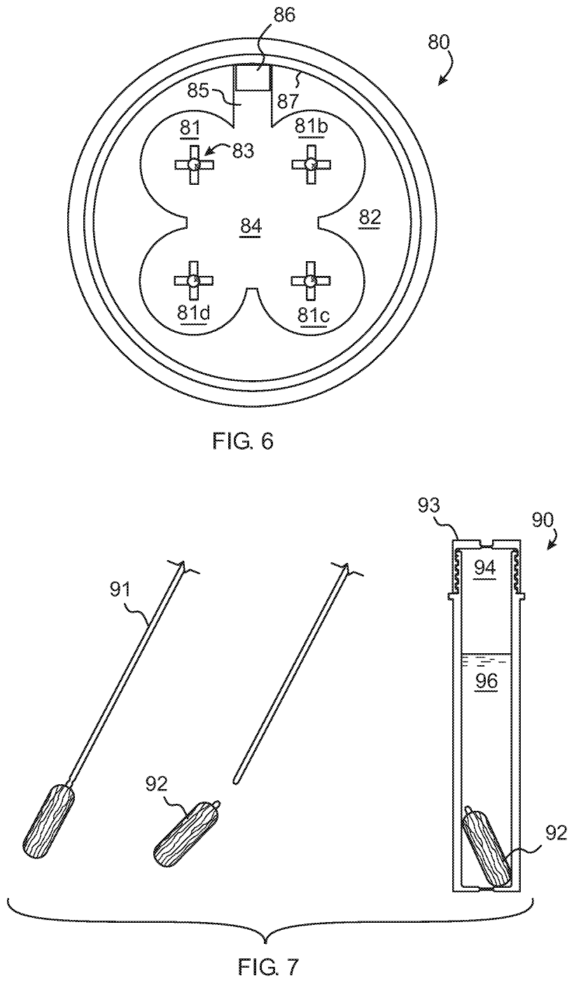

Referring now to FIGS. 6-7, there is shown an alternate embodiment of the vessel 80 adapted to conduct a single test on four combined specimens. An internal framework 82 occupies part of the internal chamber of the vessel to create four, uniformly, angularly spaced apart compartments 81,81b,81c,81d each shaped and dimensioned to secure a collection vial therein. Similar to the previous embodiments, each compartment can have a standoff 83 including pintles and spacing in alignment with the drain of its respective vial. The compartments can empty into a common basin 84 in the bottom of the vessel. The basin can feed a channel 85 leading to a cartridge 86 containing a test strip can be positioned against the substantially transparent inner side wall 87 of the vessel in order to read the test result.

Each vial 90 can be formed according to the earlier described embodiments. However, in this embodiment, a specimen collector integral with the stopper need not be used to collect a specimen. Instead, a swab stick 91 of the type commonly used to collect mucous from deep within the nasal sinuses can be employed. After the nasal mucous specimen has be collected the spongeous tip 92 can be snapped off and inserted into the vial. The stopper 93 is then affixed to the upper open end 94 of the vial. Alternately, a stopper having a specimen collector having a swab tip of the type used to collect nasal mucous or saliva can be used. Alternately, a stopper having a specimen collector of the type described in the embodiment of FIG. 2 can be used to poke the tip of the swab into the solution 96 contained in the vial. Therefore the stopper can be provided with or without a collector. Once the vials contain specimen, they can be loaded into the vessel and stored or tested in a manner consistent with the earlier embodiments. It shall be noted that the device can accomplish simultaneous opening of the drains and holes in all the vials so that proper mixing of the specimens can occur prior to contact with the strip or strips.

In this way, a single rapid screening test can be conducted which tests multiple specimens together. If a positive result is read, the four donors can then be retested individually. In this way, a more rapid screening can be conducted which can be particularly advantageous in a pandemic setting where a very large number of individuals are tested and a very high percentage of those tests are negative.

It shall be understood that the above described arrangement of elements allows for the manufacture of the components using simple injection molding techniques from common materials such as PTFE plastics and a minimum amount of assembly which can be readily automated. Indeed, with respect to the lid, vessel, stoppers, vials, and insert can each be made from a single, unitary injection molded or 3D printed piece of material.

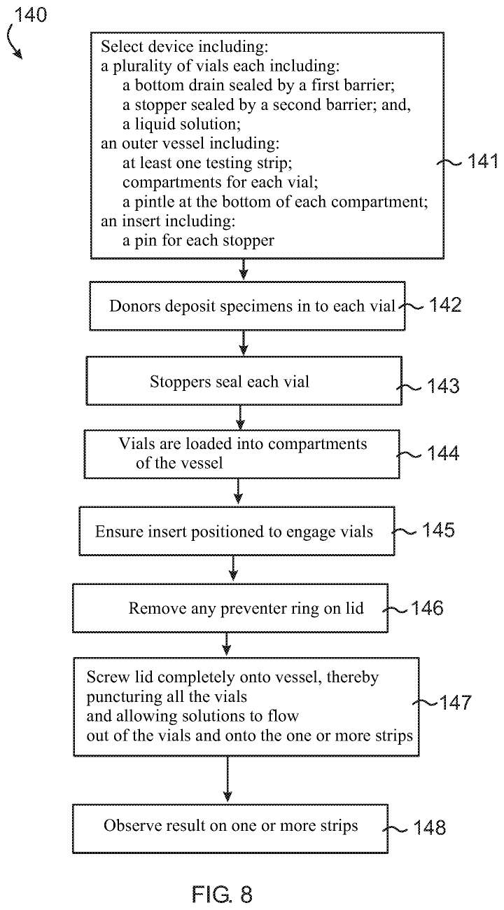

Referring now to FIG. 8, there will be described the method 140 for simultaneously conducting a preliminary screening test of a plural number of specimens. In this embodiment of the method, the device is configured for collecting and testing four fecal specimens. The method can include selecting 141 a device including: a lid, an outer vessel having a top opening, a lateral wall and a closed bottom enclosing an inner chamber that can contain a plural number of testing strips, a plural number of vials, each having a stopper having an openable barrier and a bottom drain sealed by a frangible barrier, and a vial puncturing insert. The vessel can have a compartment for each vial having a bottom pintle, and the insert can include a pin for each vial. A separate specimen is then deposited 142 by typically separate donors into each vial by inserting a collector carrying specimen into the vial and sealing it with its stopper 143. A separate vial containing a separate specimen is loaded 144 into each compartment of the vessel. If not already rotatively mounted to the lid, the insert is inserted into the vessel atop the vials so that the pins of the insert align with the sealed holed on the vial stoppers. In this way it is ensured 145 that the insert is properly positioned to engage the vials. Next, the preliminary screening is initiated by first removing 146 any preventer structure such as a base ring from the lid. The lid can then be rotated manually 147 until it is screwed fully onto the brim of the vessel, sealing its top opening and simultaneously puncturing all the vials, allowing the solutions in the vials to partially flow out of the vials and into their respective compartments. The puncturing of the vials can include moving the vials axially onto the pintles breaking the frangible barriers sealing the drains, and driving the pins of the insert onto the vials to break the openable barriers on the stoppers. The driving of the pins of the insert can include sliding a lower planar surface of the lid against an upper planar surface of the insert. Once an adequate amount of time has passed, the results of the preliminary screening tests can be observed 148 on the strips.

While the exemplary embodiments of the invention have been described, modifications can be made and other embodiments may be devised without departing from the spirit of the invention and the scope of the appended claims.

What is claimed is:

1. A self-contained device for collecting, transporting and analyzing a plurality of biological specimens, said device comprises:
an outer vessel having a top opening, a lateral wall and a closed bottom;
a hand-manipulable lid releasably sealing said top opening;
at least one signaling element contained within said vessel;
a plurality of pintles extending upwardly from said closed bottom;
a plurality of vials housed inside said outer vessel;
wherein a first one of said vials contains a first liquid specimen;
wherein a second one of said vials contains a second liquid specimen;
an insert contained within said vessel;
wherein said insert pushes all of said vials onto said pintles upon manipulation of said lid, thereby substantially simultaneously puncturing all of said vials and releasing at least one of said liquid specimens onto said at least one signaling element.

2. The device of claim 1, which further comprises:
said first one of said vials comprising:
an open upper end;
a stopper releasably sealing said open upper end;
a hole in said stopper and a frangible barrier closing said hole;
a bottom end;
a drain in said bottom end and a removable barrier closing said drain; and
a first one of said pintles being oriented to break said removable barrier upon manipulation of said lid.

3. The device of claim 2, which further comprises:
a plurality of pins extending downwardly from said insert;
wherein a first one of said pins is oriented to break said frangible barrier upon manipulation of said lid.

4. The device of claim 1, wherein said lid comprises a protrusion having a substantially planar lower surface bearing against a substantially planar upper surface of said insert, whereby said lid can rotate with respect to said insert while driving said insert axially.

5. The device of claim 1, which further comprises a framework within said vessel defining a plurality of compartments, wherein each compartment can carry a single one of said vials.

6. The device of claim 3, wherein said first one of said vials is coaxially aligned with a first one of said pins and a first one of said pintles.

7. The device of claim 1, wherein said first one of said vials further comprises:
a specimen collector projecting from an under portion of said stopper down to a lower region of said first one of said vials.

8. The device of claim 1, wherein said lid comprises a disableable preventer structure preventing said lid from being fully screwed upon said vessel, and thereby preventing initiation of any puncturing of said vials until said preventer structure is disabled.

9. The device of claim 1, wherein said first one of said pintles comprises a standoff positioned, shaped and dimensioned to keep a surface section of said closed bottom apart from a surface section of said first one of said vials.

10. A specimen collection and examination device which comprises:
a first vial comprising a first specimen;
a second vial comprising a second specimen;
a vessel nesting said first and second vials, said vessel having an open top and a closed bottom;
at least one signaling element held in said vessel;
a lid sealing said open top; and,
a puncturing structure within said vessel;
wherein said puncturing structure punctures said vials upon manipulation of said lid.

11. The device of claim 10, which further comprises:
said first vial comprising:
a first open upper end and a first closed bottom end;
a first stopper shaped and dimensioned to seal said first upper end;
said first bottom end having a first aperture and a first removable barrier sealing said first aperture;
said second vial comprising:
a second open upper end and a second closed bottom end;
a second stopper shaped and dimensioned to seal said second upper end;
said second bottom end having a second aperture and a second removable barrier sealing said second aperture; and,
wherein said puncturing structure comprises at least two pintles extending upwardly from said closed bottom in alignment with said first and second apertures.

12. The device of claim 11, which further comprises:
an insert contained within said vessel;
said first stopper comprising a first hole and a first frangible barrier sealing said first hole;
said second stopper comprising a second hole and a second frangible barrier sealing said second hole;
a first pin and a second pin projecting from an under section of said insert in alignment with said first and second holes.

13. The device of claim 12, wherein said frangible barriers and said removable barriers are simultaneously broken by manipulation of said lid.

14. The device of claim 10, which further comprises a standoff positioned, shaped and dimensioned to keep an outer surface section of said closed bottom apart from an inner surface section of said first vial.

15. A method for simultaneously analyzing a plurality of biological specimen containers, said method comprises:
selecting a vessel having a top opening, a lateral wall and a closed bottom enclosing an inner chamber;
loading a plurality of specimen containers into said inner chamber;
rotating a hand-manipulable threaded lid onto said top opening;
wherein said rotating comprises:
puncturing simultaneously said specimen containers; and,
allowing a flow of liquid solutions carrying specimens onto one or more test signaling elements.

16. The method of claim 15, wherein said puncturing comprises:
moving said specimen containers axially onto a plurality of pintles within said inner chamber; and
driving an insert having a plurality of pins axially against said specimen containers.

17. The method of claim 16, wherein said driving comprises:
sliding a lower planar surface of said lid against an upper planar surface of said insert.
